(19) United States
(12) Patent Application Publication
(10) Pub. No.: US 2018/0098875 A1
(43) Pub. Date: Apr. 12, 2018

(54) DEFECATION TREATMENT DEVICE

(71) Applicant: Gap Hwan PARK, Gyeongsannam-do (KR)

(72) Inventor: Gap Hwan PARK, Gyeongsannam-do (KR)

(21) Appl. No.: 15/573,574

(22) PCT Filed: May 13, 2016
(86) PCT No.: PCT/KR2016/005096

§ 371 Date: Nov. 13, 2017

(30) Foreign Application Priority Data
May 13, 2015 (KR) 10-2015-0066403

(51) Int. Cl. A61F 5/44; A61G 9/00

(52) U.S. Cl. CPC A61F 5/4404; A47K 11/06; A61G 9/00

(57) ABSTRACT

A defecation treatment device comprises a discharge means and an excrement containing means. The discharge means comprises a discharge tube, which has an excrement inlet formed on an end thereof such that the end is inserted into the anus, thereby sucking excrement. The excrement containing means comprises a flexible pack, which is coupled to the other end of the discharge tube so as to contain the excrement sucked into the discharge tube, a case, which contains the pack, which is coupled to the other end of the discharge tube, and which has an air outlet formed thereon so as to discharge inner air to the outside such that excrement can be contained in the pack, and a pump capable of discharging air in the case through the air outlet.

### BACKGROUND

The present invention relates to a defecation treatment device, and more particularly, to a defecation treatment device, which has a simple structure, thereby being easy to wear and to use and facilitate collection and disposal of defecation.

In general, in case of serious or critical patients, who are unconscious or cannot move around even though they are conscious, caregivers as well as the patients experience great difficulty in disposing of urine and feces.

Korean Patent No. 10-1093444 discloses an automatic defecation treatment device for patients lying in a sickbed, which is mounted on a patient through a diaper worn between the patient's legs to receive and dispose of defecation. Korean Patent No. 10-1292812 discloses an automatic defecation treatment device, which is mounted to wrap the rear part of a human's excretion part so as to automatically dispose of urine and feces on a bed. Additionally, Korean Utility Model Registration No. 20-0453814 discloses a mattress having an automatic defecation treatment device.

Such conventional defecation treatment devices have several disadvantages in that a patient needs a caregiver' help to wear the defecation treatment device because the defecation treatment device is worn between the patient's legs or is a bed having the defecation treatment device, which is worn on the patient's body and in that excretion may be stained on a mattress or patient clothes because excretion may leak out when the patient moves. In addition, the conventional defecation treatment devices have another disadvantage in that it is difficult to keep in safety or to move to another place when the patient moves to another place inevitably.

### SUMMARY OF THE INVENTION

Accordingly, the present invention has been made in an effort to solve the above-mentioned problems occurring in the prior arts, and it is an object of the present invention to provide a defecation treatment device, which has a simple structure, thereby being easy to wear and to use and facilitate collection and disposal of defecation.

To achieve the above objects, the present invention provides a defecation treatment device including a discharge means and an excrement receiving means. The discharge means has an excrement inlet formed at one end thereof and a discharge tube for inhaling excrement when the end of the discharge means is inserted into the anus. The excrement receiving means includes a flexible pack, which is combined to the other end of the discharge tube so as to receive the excrement inhaled into the discharge tube, a case, which receives the pack, which is coupled to the other end of the discharge tube, and which has an air outlet formed thereon so as to discharge the inside air of the case to the outside such that excrement can be contained in the pack, and a pump capable of discharging the inside air of the case through the air outlet.

Moreover, in the defecation treatment device, the pack is detachably combined to the other end of the discharge tube.

Furthermore, in the defecation treatment device, the case includes a body, in which the pack is contained, and a cap, which is detachably combined to the body to cover the body and is combined to the discharge tube, and, which has an air outlet.

Additionally, in the defecation treatment device, the case includes a lower body, and an upper body, which is detachably combined to one side of the lower body and seals the lower body to contain the pack.

In addition, in the defecation treatment device, the case is configured in such a way that the other side of the lower body is combined with the other side of the upper body to be able to rotate.

Moreover, in the defecation treatment device, the discharge means further comprises a separation preventing protrusion, which protrudes on the outer circumferential surface of the discharge tube to be supported inside the anus, such that the discharge tube inserted into the anus is not separated from the anus.

Furthermore, in the defecation treatment device, the discharge means further comprises a discharge valve to open and close the other end of the discharge tube.

Additionally, the defecation treatment device further includes an enema nozzle inserted into the anus through the discharge tube to inject an enema.

According to the present invention, a defecation treatment device, which includes the discharge tube and the excrement receiving means, thereby simplifying the structure of the defecation treatment device and making wearing and use of the defecation treatment device easy.

Moreover, according to the present invention, the defecation treatment device, in which the excrement receiving means is detachably attached to the discharge means, facilitates disposal of excrement.

Furthermore, according to the present invention, the defecation treatment device, which is usable after replacing only the pack for receiving excrement, can reduce the cost.

### BRIEF DESCRIPTION OF DRAWINGS

FIG. 1 is a conceptual diagram of a defecation treatment device according to a preferred embodiment of the present invention.

FIG. 2 is a conceptual diagram showing a state where a body of a case according to the preferred embodiment of the present invention illustrated in FIG. 1 is separated.

FIG. 3 is a conceptual diagram of an excrement receiving means according to the preferred embodiment of the present invention illustrated in FIG. 1.

FIG. 4 is a conceptual diagram of a defecation treatment device according to another preferred embodiment of the present invention.

FIG. 5 is a conceptual diagram of an excrement receiving means according to the preferred embodiment of the present invention illustrated in FIG. 4.

FIG. 6 is a conceptual diagram of a defecation treatment device according to a further preferred embodiment of the present invention.

List of Reference Numerals

| 3: One end | | 11: Discharge tube | |
|---|---|---|---|
| 12: Excrement inlet | | | |
| 13: Separation preventing protrusion | | | |
| 15: Discharge valve | | 20: Excrement receiving means | |
| 21: Pack | 23: Case | 24: Body | 25: Cap |
| 26: Air outlet | 27: Through hole | | |

| | | |
|---|---|---|
| 28: Body joining part | 29: One end | 30: Fixing groove |
| 33: Lower body | 34: One side | 35: Upper body |
| 36: One side | 39: Pump | 40: Enema nozzle |

### DETAILED DESCRIPTION OF THE INVENTION

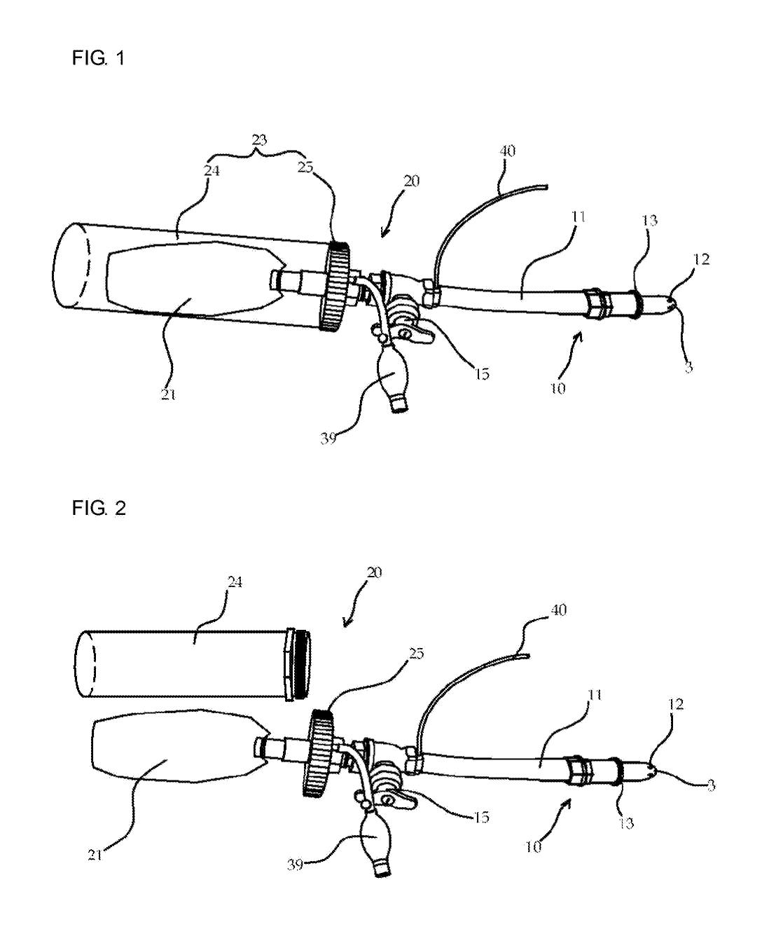

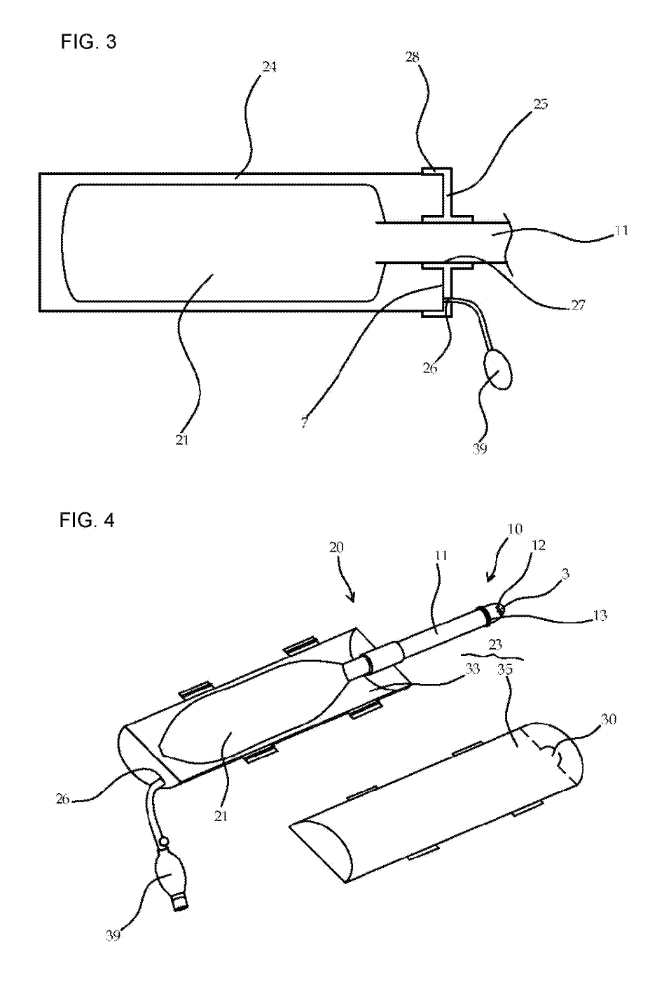

Referring to FIGS. 1 to 3, a defecation treatment device according to a preferred embodiment of the present invention will be described.

The defecation treatment device according to the present invention includes a discharge means 10, an excrement receiving means 20, and an enema nozzle 40.

The discharge means 10 includes a discharge tube 11, a separation preventing protrusion 13, and a discharge valve 15.

The discharge tube 11, whose one end is inserted into a patient's anus, serves to inhale excrement. For this, one end 3 of the discharge tube 11 is curved to be easily inserted into the anus, and the discharge tube 11 includes an excrement inlet 12 through which excrement is inhaled. When the patient defecates, excrement is inhaled into the discharge tube 11 through the excrement inlet 12.

The separation preventing protrusion 13 is a protrusion formed on the outer circumferential surface of the discharge tube 11, such that the discharge tube 11 inserted into the anus can be supported inside the anus without being separated from the anus.

The discharge valve 15 serves to open and close the discharge tube 11.

The excrement receiving means 20 includes a pack 21, a case 23 and a pump 39.

The pack 21 is detachably combined to the other end of the discharge tube 11 to receive excrement inhaled into the discharge tube 11, and may be a flexible pack. In the case that the pack 21 is the flexible pack, the pack is reduced in volume and is in a foldable state when there is no contents inside the pack, and so, it is easy to store it. In this embodiment, when the pack is detachably combined to the discharge tube 11 and defecation is complete, the pack 21, which is removed from the discharge tube 11 and receives the excrement, can be handled easily.

The case 23 is detachably combined to the other end of the discharge tube 11 to receive the pack 21 containing the excrement inhaled into the discharge tube 11. For this, the case 23 includes a body 24 and a cap 25.

The body 24 receives the pack 21 containing the excrement.

The cap 25 has an air outlet 26 for discharging the inside air of the case 23 to the outside so that the excrement can be accommodated in the pack 21, and a through hole 27 perforated in the middle part of the cap 25. Moreover, the cap 25 further has a body joining part 28 protruding on one side 5 along the peripheral portion thereof. The through hole 27 is perforated such that the discharge tube 11 is inserted into the pack 21 accommodated in the body 24 so as to be combined with the pack 21. Therefore, the through hole 27 is combined with the discharge tube 11. The body joining part 28 is screw-coupled with the body 24.

The pump 39 is mounted at the air outlet 26 formed in the cap 25 to discharge the inside air of the case 23. When the inside air of the case 23 is discharged out by the pump 39, the inside of the case 23 is in a negative pressure state, such that the excrement can be induced into the pack 21 to promote defecation.

The enema nozzle 40 is inserted into the anus through the discharge tube 11 to inject an enema. The enema is used for the purpose of treatment or when the patient cannot do defecation by himself or herself.

In this embodiment, when the discharge tube 11 is inserted into the anus, the separation preventing protrusion 13 is supported inside the anus so as to prevent the discharge tube 11 from being separated from the anus. At the time of defecation, the excrement is inhaled into the discharge tube 11 through the excrement inlet 12, and the discharge valve 15 opens the discharge tube 11, such that the excrement can be received into the pack 21 combined to the other end of the discharge tube 11. In this instance, when the inside air of the case 23 is discharged out using the pump 39 mounted at the air outlet 26, the inside of the case 23 is in the negative pressure state, such that the excrement is easily contained into the pack 21 to promote defecation.

Furthermore, because the pack 21 is detachably combined to the discharge tube 11, when defecation is complete, the pack 21 is separated from the discharge tube 11, and then, only the pack 21, in which the excrement is contained, is removed. In this instance, when a caregiver closes the discharge valve 15, it is prevented that the excrement leaks out of the discharge tube 11, and the pack 21 can be replaced with a new one for defecation. Therefore, the defecation treatment device according to the embodiment of the present invention can be used cleanly and reduce replacement costs.

Additionally, for the purpose of treatment or when the patient cannot do defecation by himself or herself, after the discharge tube 11 is inserted into the anus, the enema nozzle 40 is inserted through the discharge tube 11 so as to inject an enema. When the enema is injected through the enema nozzle 40, defecation is active, and so, the patient can defecate. In this instance, the caregiver opens the discharge valve 11, such that excrement is accommodated in the pack 21. After the enema is injected, it is preferable to inject the enema through the enema nozzle 40 after inserting the discharge tube 11 into the anus because defecation may be started before the discharge tube 11 is inserted into the anus due to power applied to the anus.

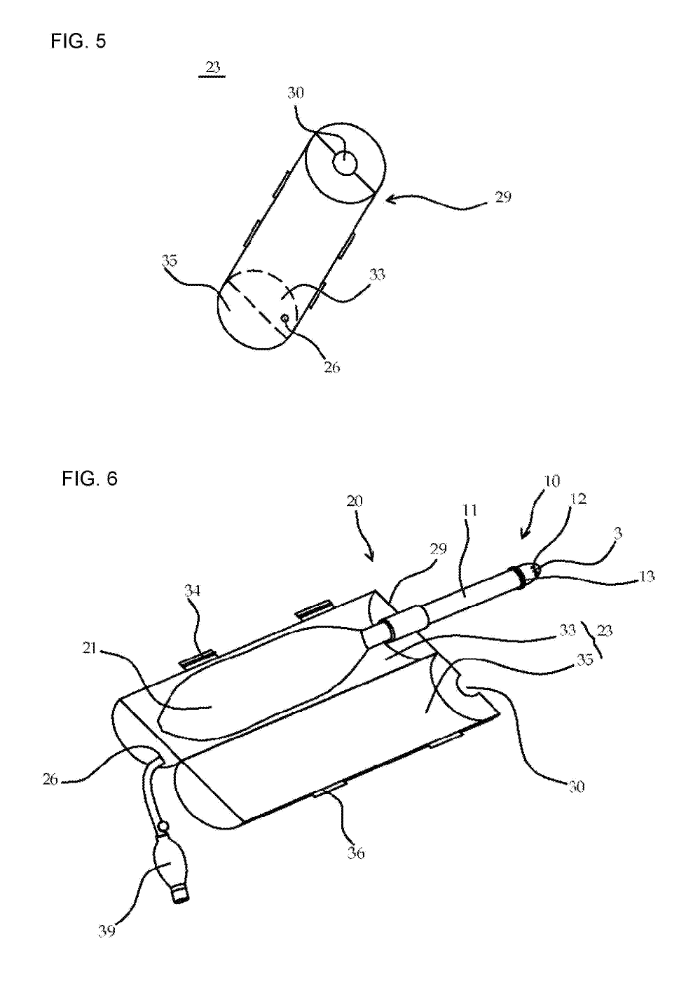

FIGS. 4 and 5 are defecation treatment devices according to other preferred embodiments of the present invention. Referring to FIGS. 4 and 5, the defecation treatment devices according to other preferred embodiments of the present invention will be described.

The pack 21 of the excrement receiving means 20 is combined to the other end of the discharge tube 11 to be formed integrally with the discharge tube 11. So, when defecation is complete, the pack 21, in which excrement is contained, can be promptly handled together with the discharge tube 11 without separating the pack 21 from the discharge tube 11. Moreover, the defecation treatment device according to this embodiment can reduce inconvenience to wash the discharge tube 11 after the pack 21 is separated from the discharge tube 11.

The case 23 accommodates the pack 21 therein, and includes a fixing groove 30, which is formed at one end 29 to be matched with the discharge tube 11 in shape such that the discharge tube 11 combined with the pack 21 is fixed, and an air outlet 26 formed at the other end. For this, the case 23 includes a lower body 33 and an upper body 35.

The lower body 33 and the upper body 35 are detachably combined to each other. When the lower body 33 and the upper body 35 are combined after the discharge tube 11 is inserted therebetween, the inside of the case 23 is sealed. In this embodiment, it is easy to receive and remove the pack 21 because the case 23 is divided into the lower body 33 and the upper body 35. Detailed description of the remaining components will be omitted because they are the same as the embodiment illustrated in FIG. 1.

FIG. 6 is a defecation treatment device according to another preferred embodiment of the present invention. Referring to FIG. 6, the defecation treatment device according to another preferred embodiment of the present invention will be described.

In FIG. 6, one side 34 of the lower body 33 and one side 36 of the upper body 35 are detachably combined to each other, and the other side of the lower body 33 and the other side of the upper body 35 are combined to each other to be able to rotate. That is, the other side of the upper body 35 and the other side of the lower body 33 are hinge-coupled with each other, such that the pack 21 can be accommodated into or removed from the case 23 by one side 36 of the upper body 35 and one side 34 of the lower body 33, which are detachably combined to each other.

1. A defecation treatment device comprising:
an discharge means having an excrement inlet formed at one end thereof and a discharge tube for inhaling excrement when the end of the discharge means is inserted into the anus; and
an excrement receiving means having a flexible pack, which is combined to the other end of the discharge tube so as to receive the excrement inhaled into the discharge tube, a case, which receives the pack, which is coupled to the other end of the discharge tube, and which has an air outlet formed thereon so as to discharge the inside air of the case to the outside such that excrement can be contained in the pack, and a pump capable of discharging the inside air of the case through the air outlet.

2. The defecation treatment device according to claim 1, wherein the pack is detachably combined to the other end of the discharge tube.

3. The defecation treatment device according to claim 2, wherein the case comprises a body, in which the pack is contained, and a cap, which is detachably combined to the body to cover the body and is combined to the discharge tube, and, which has an air outlet.

4. The defecation treatment device according to claim 2, wherein the case comprises a lower body, and an upper body, which is detachably combined to one side of the lower body and seals the lower body to contain the pack.

5. The defecation treatment device according to claim 4, wherein the case is configured in such a way that the other side of the lower body is combined with the other side of the upper body to be able to rotate.

6. The defecation treatment device according to claim 1, wherein the discharge means further comprises a separation preventing protrusion, which protrudes on the outer circumferential surface of the discharge tube to be supported inside the anus, such that the discharge tube inserted into the anus is not separated from the anus.

7. The defecation treatment device according to claim 6, wherein the discharge means further comprises a discharge valve to open and close the other end of the discharge tube.

8. The defecation treatment device according to claim 8, further comprising an enema nozzle inserted into the anus through the discharge tube to inject an enema.
